(19) United States
(12) Patent Application Publication
(10) Pub. No.: US 2024/0366705 A1
(43) Pub. Date: Nov. 7, 2024

(54) COMPOSITIONS COMPRISING METAL-COMPLEXED PLANT COMPOUNDS AND USES THEREOF

(71) Applicant: RD Life Sciences, LLC, Largo, FL (US)

(72) Inventors: Daniel J. DuBourdieu, Limerick, ME (US); Rajiv Lall, Clearwater, FL (US); Jamil Talukder, Kenneth City, FL (US)

(73) Assignee: RD Life Sciences, LLC, Largo, FL (US)

(21) Appl. No.: 18/598,827

(22) Filed: Mar. 7, 2024

Related U.S. Application Data
(60) Provisional application No. 63/500,334, filed on May 5, 2023.

(51) Int. Cl. A61K 36/45; A61K 33/30; A61P 29/00

(52) U.S. Cl. CPC A61K 36/45; A61K 33/30; A61P 29/00

(57) ABSTRACT

The invention is directed to compositions that include metal-complexed plant compounds. The compositions can be derived from liquid concentrates, extracts, and pomace from plants, such as fruits or vegetables. The compositions can further include additional nutraceutical antimicrobials, antioxidants, and immune boosters. The compositions can be administered to animals to increase animal health, reduce stress, reduce oxidative stress, maintain health, improve health, improve immunity, improve vaccination efficacy, increase feed conversion rate, decrease pro-inflammatory cytokine levels, increase anti-inflammatory cytokines, or any combination thereof in an animal. The compositions of the invention exhibit enhanced bioavailability relative to control compositions that do not contain the metal-complexed compounds.

### FIELD OF THE INVENTION

The present invention is directed to compositions comprising metal-complexed plant compounds and uses thereof, such as for maintaining and improving health and/or boosting immunity in animals.

### BACKGROUND

Many health issues in animals and humans can be improved by supporting and having a healthy immune system. On the whole, the immune system does a remarkable job of defending against disease-causing microorganisms and other sources of stress on the immune system. Bacteria and flu viruses are sources of disease, but less obvious sources of stress on the immune system including simply being old, traveling, or having emotional issues can also be a source of stress. As good as the immune system is, it sometimes fails, a germ invades successfully, and sickness occurs. Prevention and treatment of infections by the use of natural products instead of pharmaceutical antibiotics has gained traction over the last decade as a means to prevent antibiotic resistance. The use of cranberries for treating urinary tract infections is an example of one such natural product. Cranberries contain numerous active ingredients that work to reduce infections through a multitude of actions. The antimicrobial actions of cranberries have been examined for their use in animal feeds as an alternative to antibiotics, to act as growth promotors in production animals. The use of cranberries as an alternative to antibiotics in animal production relies on polyphenolic compounds found in cranberries that can act as antioxidants to combat oxidative stress due to the stress of various infections. Various infective agents can cause the production of free radicals and oxidative stress that, in turn, causes problems for cells, and this damage can spread throughout the body. For example, oxygen free radicals can react with cellular components to damage cells membranes, proteins, and DNA. Antioxidants combat the free radicals by donating an electron to the free radical of reactive oxygen species. Antioxidants are molecules that prevent free radicals from taking electrons and causing cellular damage. Antioxidants are able to give an electron to a free radical without becoming destabilized because they are inherently stable without having a complete electron valence shell. As such, antioxidants can stop free radical chain reactions and limit the damage to the body. The body has some inherent antioxidants such as glutathione, but obtaining antioxidants from the diet or supplements can help reduce oxidative stress and help the immune system. Once the balance between the accumulation of reactive oxidative species and the removal of oxides is broken, oxidative stress is established. The ultimate consequences of this oxidative stress include tissue damage, inflammation responses, and cell death.

A strategy to improve general health in humans and animals would be to help reduce oxidative stress and also help the immune system in other more specific ways.

### SUMMARY OF THE INVENTION

One aspect of the invention is directed compositions comprising a metal-complexed plant compound comprising a plant compound complexed with a metal. Such compositions are referred to herein as “complexed compositions.” In some versions, the metal-complexed plant compound is generated by a process comprising mixing the metal with a semi-purified plant composition that comprises the plant compound to thereby complex the plant compound with the metal and generate the metal-complexed plant compound.

In some versions, the semi-purified plant composition comprises a plant pomace, a plant juice, a liquid plant concentrate, a plant extract, or a combination thereof. In some versions, the semi-purified plant composition is derived from a fruit or vegetable.

In some versions, the plant compound comprises a polyphenol. In some versions, the plant compound comprises a flavonoid.

In some versions, the metal is a divalent metal or a trivalent metal. In some versions, the metal is zinc, iron (III) or a combination thereof. In some versions, the metal is mixed with the semi-purified plant composition in an amount of at least 0.1% w/w of the semi-purified plant composition.

In some versions, the process comprises mixing the metal with the semi-purified plant composition in a solvent. In some versions, the solvent comprises water, an alcohol, or a combination thereof.

In some versions, the complexed composition further comprises an active ingredient extraneous to the semi-purified plant composition. In some versions, the active ingredient comprises at least one of an antioxidant, an antimicrobial agent, and an immune-boosting agent. In some versions, the active ingredient comprises one or more of a phytobotanical, an anthocyanin, a polyphenol, ferulic acid, N-acetyl cysteine, a Spirulina extract, a clay, and styrene-divinylbenzene resin.

In some versions, the complexed composition is in the form of a powder.

Other aspects of the invention are directed to methods of administering a complexed composition of the invention to an animal.

In some versions, the animal is a human, a production animal, or a companion animal. In some versions, the complexed composition is administered to the animal in an amount effective to increase weight of the animal relative to a control animal not administered the complexed composition.

In some versions, the complexed composition is administered to the animal in an amount effective to decrease the feed conversion rate of the animal relative to a control animal not administered the complexed composition.

In some versions, the complexed composition is administered in an amount effective to decrease serum levels of at least one pro-inflammatory cytokine or increase serum levels of at least one anti-inflammatory cytokine in the animal relative to a control animal not administered the complexed composition. In some versions, the complexed composition is administered in an amount effective to decrease serum levels of at least one of Il-1 beta, IL-2, IL-8, IL-7, IL-15, IL-18, MCP-1, TNFα, in the animal relative to a control animal not administered the complexed composition. In some versions, the complexed composition is administered in an amount effective to decrease at least one of interleukin (IL)-1 receptor antagonist, IL-4, IL-6, IL-10, IL-11, and IL-13.

In some versions, the animal is an animal suffering from a condition comprising one or more of iron anemia, cancer, arthritis, an inflammatory condition, a cardiovascular condition, a neurological condition, a pulmonary condition, diabetes, a hepatic condition, a gastrointestinal condition, a viral disease, a prion disease, mycotoxin infection, and an infectious disease.

In some versions, the complexed composition is administered orally to the animal.

The objects and advantages of the invention will appear more fully from the following detailed description of the preferred embodiment of the invention made in conjunction with the accompanying drawings.

### BRIEF DESCRIPTION OF THE DRAWINGS

FIG. 1. General structure of flavonoids.

FIG. 2. Anthocyanidin and anthocyanin structures.

FIG. 3. General structure of flavonols.

FIG. 4. Structures of common polyphenolics found in cranberries: (a) cyanidin; (b) peonidin; (c) quercetin; (d) myricetin; (e) procyanidin dimer exhibiting a B-type linkage (4β→8); (f) procyanidin dimer exhibiting an A-type linkage (4β→8, 2β→O→7).

FIG. 5A. Chemical structure of procyandin.

FIG. 5B. Chemical structure of some polyphenols. When R═H, it is a procyanidin: catechin (R₁═H and R₂═OH) and epicatechin (R₁═OH and R₂═H); When R═OH, it is a prodelphinidin: gallocatechin (R₁═H and R₂═OH) and epigallocatechin (R₁═OH and R₂═H).

FIG. 6. Flavonol-metal complexes.

FIG. 7. Anthocyanidin-metal complexes.

FIG. 8. UV-vis profiles of wet cranberry pomace and wet cranberry pomace-zinc chelated in water.

FIG. 9. UV-vis profiles of wet cranberry pomace and wet cranberry pomace-zinc chelated in methanol.

FIG. 10. UV-vis profiles of dried cranberry pomace control powder and dried cranberry pomace powder chelated with zinc in methanol.

FIG. 11. UV-vis profiles of dried cranberry pomace control powder and dried cranberry pomace powder chelated to zinc in water.

FIGS. 12A and 12B. Photodiode array (PDA) (FIG. 12A) and high performance liquid chromatography (HPLC) profiles (FIG. 12B) of liquid cranberry concentrate control.

FIGS. 13A and 13B. PDA (FIG. 13A) and HPLC profiles (FIG. 13B) of liquid cranberry concentrate-zinc chelation complexes.

FIGS. 14A and 14B. Control liquid cranberry concentrate mass spectrum profile. Only the portions of the x-axis having peaks are shown.

FIGS. 15A and 15B. 10% Zinc-cranberry liquid concentrate mass spectrum profile. Only the portions of the x-axis having peaks are shown.

FIGS. 16A and 16B. 50% Zinc-cranberry liquid concentrate mass spectrum profile. Only the portions of the x-axis having peaks are shown.

FIG. 17. UV profiles of FeCl₂ and FeCl₃ chelation to control liquid cranberry concentrates.

FIG. 18. HPLC chicken serum profiles of cranberry-zinc chelate 1 hour post oral delivery (upper trace), cranberry control post chicken oral delivery (middle trace), and serum without either (bottom trace).

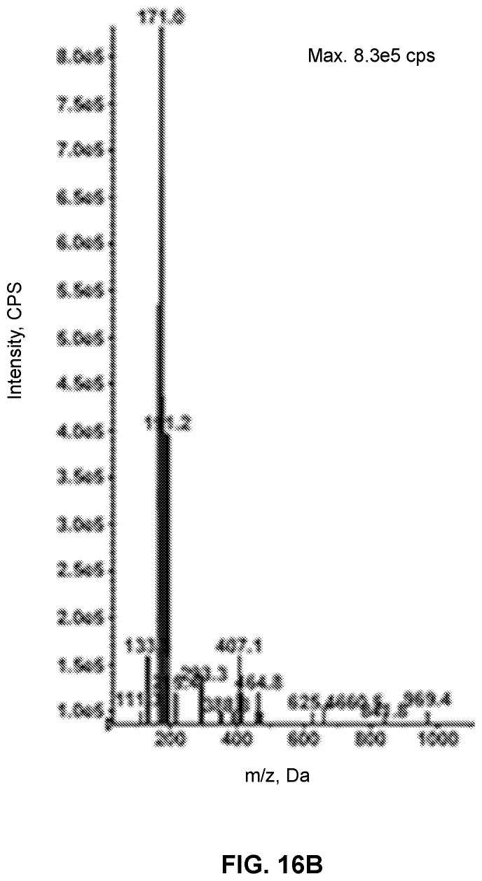

FIG. 19. Weight gain in chickens with cranberry-zinc pomace chelates compared to control.

FIG. 20. Feed conversion rate (FCR) in chickens with cranberry pomace-zinc chelates compared to control.

FIG. 21. IL-1-Beta in serum of chicken fed cranberry pomace-zinc chelates compared to control.

FIG. 22. IL-6 in serum of chickens fed cranberry pomace-zinc chelates compared to control.

FIG. 23. IFN gamma in serum of chickens fed cranberry pomace-zinc chelates compared to control.

### DETAILED DESCRIPTION OF THE INVENTION

One aspect of the invention is directed to complexed compositions comprising a metal-complexed plant compound. The metal-complexed plant compounds can comprise a plant compound complexed with a metal. The complex of the plant compound and the metal can be in the form of a chelate complex. As used herein, “complexed composition” refers to any composition comprising a metal-complexed plant compound of the invention.

The plant compound can comprise any compound found in a plant or a specific component thereof, such as a fruit, vegetable, or other type of plant or component thereof. The plant compound in some versions comprises a polyphenol. Exemplary polyphenols include flavonoids, tannic acid, and ellagitannins. Exemplary flavonoids include flavones, flavonols, flavanols, flavanones, isoflavones, proanthocyanidins, and anthocyanins. The plant compound can comprise a compound from any one or more of these compound classes.

The metal can comprise any metal. In some versions, the metal comprises a divalent metal, a trivalent metal, or a combination thereof. Exemplary divalent metals is selected from the group consisting of zinc, copper, iron (II), magnesium, cadmium selenium, titanium, and cobalt. Exemplary trivalent metals include iron (III), ruthenium, iridium, and aluminum. In some versions, the metal comprises zinc, iron (III) or a combination thereof.

The metal-complexed plant compound can be generated by a process comprising mixing the metal with a semi-purified plant composition that comprises the plant compound. The mixing complexes the plant compound with the metal and generates the metal-complexed plant compound. As used herein, “semi-purified plant composition” refers to a processed plant composition in which no single chemical compound in the composition constitutes more than 90% w/w of the dry weight of the composition. “Processed plant composition” refers to remnants from physically and/or chemically processing a plant to at least partially break down and/or change the original structure of the plant and/or to remove at least a portion of at least one component of the plant. “Single chemical compound” in this context refers to the chemical entities in a composition having the same chemical structure. “Dry weight” refers to the weight of the composition after its water content has been removed, e.g., by drying.

In various versions of the invention, the semi-purified plant composition can be such that no single chemical compound in the semi-purified plant composition constitutes more than 85% w/w, more than 80% w/w, more than 75% w/w, more than 70% w/w, more than 65% w/w, more than 60% w/w, more than 55% w/w, more than 50% w/w, more than 45% w/w, more than 40% w/w, more than 35% w/w, more than 30% w/w, more than 25% w/w, more than 20% w/w, more than 15% w/w, more than 10% w/w, more than 5% w/w, more than 1% w/w, more than 0.5% w/w, more than 0.1% w/w, more than 0.05% w/w, more than 0.01% w/w, more than 0.005% w/w, more than 0.001% w/w, more than 0.0005% w/w, or more than 0.0001% w/w of the dry weight of the semi-purified plant composition.

In some versions, the semi-purified plant composition comprises a plant pomace. “Plant pomace” as used herein refers to solid remnants of a plant or component thereof after the plant or component thereof has been pressed to remove at least some of its liquid.

In some versions, the semi-purified plant composition comprises a plant juice. “Plant juice as used herein refers to liquid extracted from a plant or component thereof by pressing the plant or component thereof.

In some versions, the semi-purified plant composition comprises a liquid plant concentrate. “Liquid plant concentrate” refers to remnants of a plant juice after water has been removed. Exemplary methods for removing water include evaporation and vacuum.

In some versions, the semi-purified plant composition comprises a plant extract. “Plant extract” refers to remnants of a plant or component thereof that have been extracted from the plant or component thereof with a solvent. Exemplary solvents include water, alcohols, and mixtures thereof, among others.

The plant or component thereof used to generate the semi-purified plant composition can comprise any plant or component thereof. In some versions, the plant or component thereof used to generate the semi-purified plant composition comprises a fruit or a vegetable. Exemplary fruits or vegetables include cranberry, orange, blueberry, grape, apple, raspberry, and olive.

The metal can be mixed with the semi-purified plant composition in any amount. In various versions of the invention, the metal is mixed with the semi-purified plant composition in an amount of 0.0001% w/w, 0.0005% w/w, 0.001% w/w, 0.005% w/w, 0.01% w/w, 0.05% w/w, 0.1% w/w, 0.5% w/w, 1% w/w, 5% w/w, 10% w/w, 15% w/w, 20% w/w, 25% w/w, 30% w/w, 35% w/w, 40% w/w, 45% w/w, 50% w/w, 55% w/w, 60% w/w, 65% w/w, 70% w/w, 75% w/w, 80% w/w, 85% w/w, 90% w/w, 95% w/w, or 99% w/w of the semi-purified plant composition. As used herein, the mass of a metal added to or included in a composition refers to the mass of the metal ion itself in the composition, and not an entire salt or other form thereof. For example, the mass of zinc in a composition which the zinc is added as ZnCl₂ would be the mass of the Zn2+ ion itself, rather than the mass of the ZnCl₂ salt added to the composition.

In some versions, the process comprises mixing the metal with the semi-purified plant composition in a solvent. Exemplary solvents include water and alcohols. Exemplary alcohols include methanol, ethanol, and propanol, among others. The solvent in some cases is an extraneously added solvent, i.e., added solvent that is not already contained by the semi-purified plant composition. The extraneously added solvent can be of a type that is the same as any solvent contained by the semi-purified plant composition or can be of a type that is different than any solvent contained by the semi-purified plant composition.

In some versions, the complexed composition further comprises an active ingredient extraneous to the semi-purified plant composition, i.e., added active ingredient that is not already contained by the semi-purified plant composition. The extraneously added active ingredient can be of a type that is the same as any active ingredient contained by the semi-purified plant composition or can be of a type that is different than any active ingredient contained by the semi-purified plant composition. In some versions, the active ingredient comprises at least one of an antioxidant, an antimicrobial agent, and an immune-boosting agent. In some versions, the active ingredient comprises one or more of a phytobotanical, an anthocyanin, a polyphenol, ferulic acid, N-acetyl cysteine, a Spirulina extract, a clay, and styrene-divinylbenzene resin (e.g., Amberlite™ XAD®-2 (Millipore Sigma)).

The complexed composition can be in any form. Exemplary forms include wet flakes, dried flakes, powders, pastes, liquids, foods, and animal feeds, among others.

Some aspects of the invention are directed to methods of administering the complexed composition of the invention to an animal.

The animal can be any type of animal. In some versions of the invention, the animal is a mammal. In some versions of the invention, the animal is a human. In some versions, the animal is a non-human animal. In some versions of the invention, the animal is a companion animal. “Companion animal” as used herein refers to a domesticated or domestic-bread non-human animal. Exemplary companion animals include canines and felines. In some versions of the invention, the animal is a production animal. “Production animal” as used herein refers to a non-human animal used for meat, fiber, milk, or other products. Exemplary production animals include bovines, avians, and porcines. In some versions of the invention, the animal is a research animal. “Research animal” as used herein refers to animals bred for research use. In some versions, the animal is an avine, a bovine, an ovine, a reptilian, a porcine, or a caprine.

The complexed composition can be orally administered to the animal, among other methods of administration.

In some versions, the complexed composition is administered to the animal in an amount effective to increase weight of the animal relative to a control animal not administered the complexed composition. In some versions, the complexed composition is administered to the animal in an amount effective to decrease the feed conversion rate of the animal relative to a control animal not administered the complexed composition. In some versions, the complexed composition is administered in an amount effective to decrease serum levels of at least one pro-inflammatory cytokine or increase serum levels of at least one anti-inflammatory cytokine in the animal relative to a control animal not administered the complexed composition. In some versions, the complexed composition is administered in an amount effective to decrease serum levels of at least one of Il-1 beta, IL-2, IL-8, IL-7, IL-15, IL-18, MCP-1, TNFα, in the animal relative to a control animal not administered the complexed composition. In some versions, the complexed composition is administered in an amount effective to decrease at least one of interleukin (IL)-1 receptor antagonist, IL-4, IL-6, IL-10, IL-11, and IL-13. In some versions, the administering can reduce stress, reduce oxidative stress, maintain health, improve health, improve immunity, improve vaccination efficacy, increase feed conversion rate, decrease pro-inflammatory cytokine levels in the animal, or any combination thereof

In some versions, the animal is an animal suffering from a condition or a disease. In some versions, the animal is an animal suffering from a condition comprising one or more of iron anemia, cancer, arthritis, an inflammatory condition, a cardiovascular condition, a neurological condition, a pulmonary condition, diabetes, a hepatic condition, a gastrointestinal condition, a viral disease, a prion disease, mycotoxin infection, and an infectious disease.

In some versions, the composition is administered daily to the animal.

Some aspects of the invention are directed to liquid fruit concentrate-metal complexes, dry and liquid fruit extract-metal complexes and fruit pomace-metal complexes. The liquid concentrates and extracts can be selected from any fruit, including cranberry, orange, blueberry, grape, apple, raspberry, and olive. The pomace can be selected from any fruit pomaces, including cranberry, orange, blueberry, grape, apple, raspberry, and olive pomaces. Particular embodiments of the invention are directed to cranberry liquid concentrate-metal complexes, cranberry extract-metal complexes, and cranberry pomace-metal complexes. The cranberry pomace-metal complexes are complexes comprising at least one of the components found in cranberry pomace complexed (e.g., chelated) to a metal. The dry cranberry extract-metal complexes are complexes comprising at least one of the components found in cranberry extract complexed (e.g., chelated) to a metal. The metal can be zinc or other metals. The complexed compositions can be combined with additional immune-boosting nutraceuticals and vitamins for increased general health. It may be combined with aflatoxin binding agents such as clays or styrene-divinylbenzene resin (e.g., Amberlite™ XAD®-2 (Millipore Sigma)) for use in production animals to increase gain.

The complexed compositions of the invention can be orally delivered to animals for the purpose of stimulating the immune system in times of oxidative stress from diseases or other causes of stress such as during growth of production animals, to help maintain general health. Exemplary animals include mammals, fish, birds, and reptiles, among others.

The complexed compositions of the invention can help animals achieve better overall health, especially in times of oxidative stress, such as from diseases or to maintain optimum immunity health on a daily basis. The metal-complexed plant compounds can be combined with other immune modulating agents to further enhance bioavailability and antioxidant capability to achieve optimal health.

The invention in some aspects encompasses a chelation of zinc or other metals to components found in cranberry liquid concentrates, dry extracts, and/or pomace or other sources of liquid concentrates, dry extracts, and/or pomace such as apple, orange, blueberry, grapes, or other fruits.

The molecular complex of the invention advances the art of stimulating the immune system by providing metal chelated flavonols, anthocyanidins, and/or proanthocyanidins simultaneously from liquid concentrates, dry extracts, and pomaces. The invention advances the art by creating liquid concentrate-metal complexes, dry extracts-metal complexes, and pomace-metal complexes that synergistically increase the antioxidant and immune-stimulating capability of all the components in pomaces that can bind metals. By having a synergistic capability, relatively less amount of the metal-complexed plant compounds of the invention are required to achieve the same efficacy for anti-oxidative, anti-inflammatory, anti-microbial and immune-boosting aspects compared to uncomplexed compounds. This allows for even safer use of liquid concentrates, extracts, pomaces, and zinc and alleviates toxicity issues that might be possible when higher doses of these individual types of molecules have been used.

The invention in some aspects is directed to complexes comprising a metal complexed (e.g., chelated) to at least one component in fruit liquid concentrates, extracts and pomaces. It is not obvious that merely semi-purified components in pomace and other processed fruit compositions will get chelated. This uncertainty is due to the competition of metals to a variety of chemical constituents found in pomaces. Unexpectedly, we discovered that at least one component gets chelated in liquid cranberry concentrates, extracts, and whole pomaces when zinc or other metals are added using either water, methanol, or other solvents to solvate the reaction. Chelation is a type of chemical bonding of ions and molecules to metal ions. It involves the formation or presence of two or more separate coordinate bonds between a polydentate (multiple bonded) ligand and a single central atom. The ionic form of the central atom can be critical to the success of chelation to the ligand, as divalent ions and trivalent ions do not always bind in the same manner. Various zinc forms can be utilized in the invention including, but not limited to, zinc chloride, zinc acetate, zinc gluconate, zinc sulfate, and zinc picolinate in anhydrous or hydrated formats. Other ionic metals can be used including, but not limited to, iron and copper in the chelation bonding mechanism.

Certain metals in the complexed compositions of the invention can boost the immune system. Zinc is one such metal of a divalent nature. The presence of zinc in the complexed compositions of the invention can be important for multiple cellular functions including immunity. The presence of zinc in the complexed compositions of the invention can be for normal development and function of cell-mediating innate immunity, neutrophils, and natural killer cell. Beneficial therapeutic responses of presence of zinc in the complexed compositions of the invention can include treating the diarrhea of children, chronic hepatitis C, shigellosis, leprosy, tuberculosis, pneumonia, acute lower respiratory tract infection, common cold, and leishmaniasis. The presence of zinc in the complexed compositions of the invention can be effective in decreasing incidences of infections in elderly patients with sickle cell disease and in decreasing incidences of respiratory tract infections in children. The presence of zinc in the complexed compositions of the invention can prevent blindness in elderly individuals with dry type of macular disorder. The presence of zinc in the complexed compositions of the invention can be effective in decreasing oxidative stress and pro-inflammatory cytokines such as TNF-a and IL-1b in elderly individuals and patients. The presence of zinc in the complexed compositions of the invention can be successfully used as a therapeutic and preventive agent for many conditions.

Zinc is considered to be relatively nontoxic, particularly if taken orally. However, manifestations of overt toxicity symptoms (nausea, vomiting, epigastric pain, lethargy, and fatigue) will occur with extremely high zinc intakes. Even lower levels of zinc supplementation, closer in amounts to the recommended daily allowance (RDA), have been suggested to interfere with the utilization of copper and iron and to adversely affect HDL cholesterol concentrations. Individuals using zinc supplements should be aware of the possible complications attendant to their use (Fosmire, 1990). As such, it is desirable to get the benefits of zinc in as low a dose as possible.

Iron is important for piglets because the most common health disorder in suckling piglets is iron deficiency anemia. Iron can be found in divalent (iron (II)) and trivalent (iron III)) formats. Suckling piglets need more iron for rapid and healthy growth than is available from sow's milk alone. Therefore, iron supplementation is common on both conventional and organic farms and is usually carried out by a single injection of 200 mg iron during the piglets' first days of life. However, the method is very stressful to the piglets. They will suffer more pain if a greater dosage of iron is given intramuscularly. Furthermore, poor iron injection techniques may cause considerable trauma to the muscles, staining of hams or create abscesses and lead to downgrading of the carcasses. As such, the use of oral delivery of iron with the complexed compositions of the invention is desirable and can be combined with other ingredients that can help the immune system and the overall health of the animal.

Aspects of the invention employ liquid fruit concentrates. Once a fruit such as cranberry is picked, the fruit is crushed and the juice is released that contains numerous flavonoids, anthocyanidins, proanthocyanidins and other polyphenolics. The liquid juice is heated to evaporate off much of the fruit's water content. Other techniques included using vacuum to remove the water. These methods significantly reduce the volume of fruit juice liquid and thus easier to transport in bulk. The resultant liquid fruit concentrate can be six times the strength of a regular juice.

Besides simple liquid concentrates of a fruit juice, there are other forms that can be manufactured from a fruit. Some of these include fruit extracts, including liquid and solid (e.g., powdered) fruit extracts. Liquid fruit extracts can be obtained by soaking the fruit material in a solvent, such as water or alcohol, to extract the active compounds. The resulting liquid extract is highly concentrated and can be used in a wide range of applications, such as food and beverages, nutraceuticals, and cosmetics. Powdered fruit extracts can be obtained by drying the liquid extract and then grinding it into a fine powder. This type of extract is easier to store and transport and can be used in various applications, such as supplements and pharmaceuticals. Freeze-dried fruit extracts can be obtained by freezing the liquid extract and then drying it under vacuum to remove the water. The resulting powder is highly concentrated and retains the full spectrum of nutrients and beneficial compounds found in the fruit.

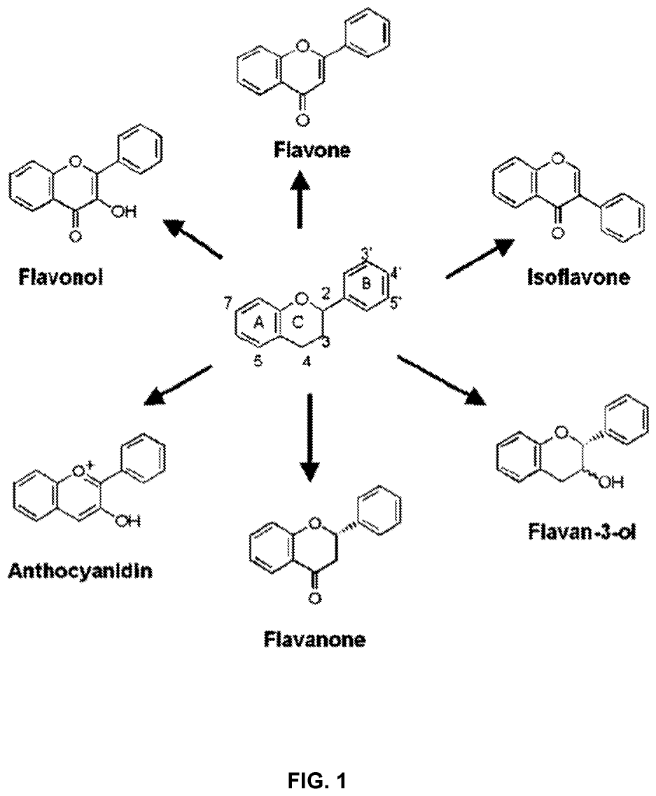

Fruits such as cranberries have been used as food for cons by humans and animals. Cranberries are considered safe when taken orally by most people. Pomaces from fruit such as olives (Ribeiro et al., 2021), apples (Skinner et al., 2018), orange (GRAS GRN-000719) are also safe. Cranberry extract powders are also generally recognized as safe by the FDA (GRAS GRN 873). Pomace comprises material left after the processing of cranberries, blueberries, and other fruits. Pomace can be composed of skin, seeds, and stems left over from pressing the fruit to obtain its juice and is considered a waste product. Industry has problems with disposing of it due to environmental problems surrounding its low pH. It also has limited use in animal feed due to its low protein content. Pomace contains a variety of flavonoid polyphenolic compounds such as anthocyanidins, flavonols, and procyanidins (White et al., 2010) that can help reduce risks of various diseases when used in purified formats. These polyphenolic structures have the potential to be utilized as antioxidants and immune booster in feeds but suffer from low bioavailability. These flavonoid compounds include ring structures with the rings designated at A, B, and C (FIG. 1).

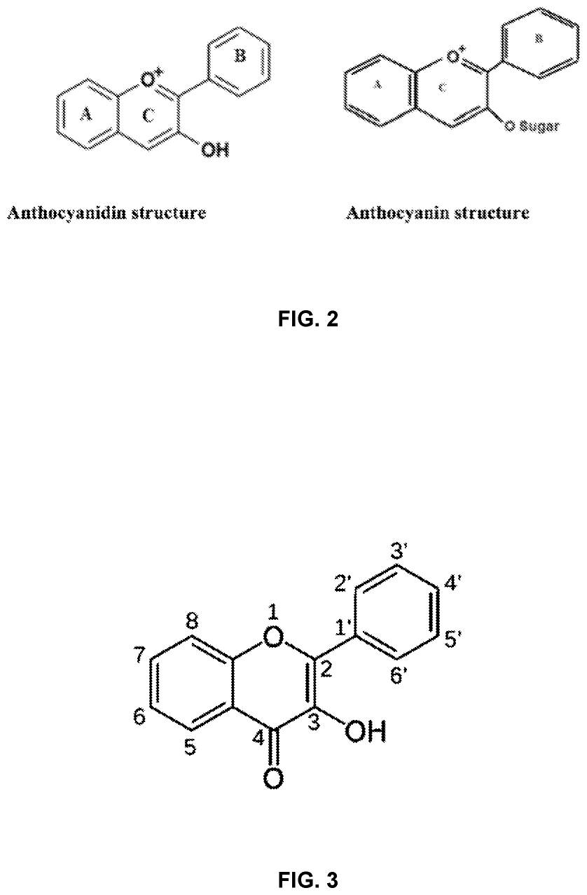

Anthocyanins include pigments found in the skins of fruits. There are six major anthocyanidins and two are commonly found in cranberries: cyanidin and peonidin. Normally they are glycosylated to various sugars in the fruit, which helps to stabilize them. The aglycone form is known as anthocyanidins (FIG. 2). The chemical structure of anthocyanidins allows the chelation of metals to them. Anthocyanins can chelate multivalent metal ions with at least two free hydroxyl groups on the B-ring. This includes, but is not limited to, Fe²⁺, Fe³⁺, Ga³⁺, Al³⁺, Mg²⁺, Sn²⁺, Ti²⁺, and Cu²⁺. Zinc chelation of anthocyanins such as cyanidin results in a stable molecule (Janceshma et al., 2020).

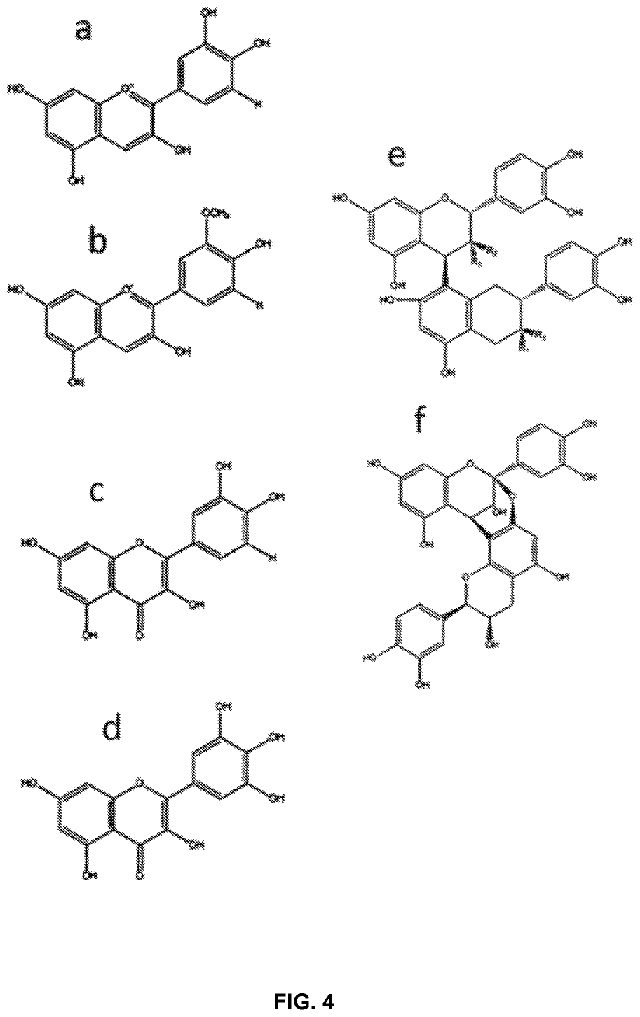

Cranberry contains flavonols which are a class of flavonoids that are characterized by a hydroxyl group at position 3, a carbonyl group at position 4, and a double bond between carbons 2 and 3 on the C ring of the flavan structure (FIG. 3).

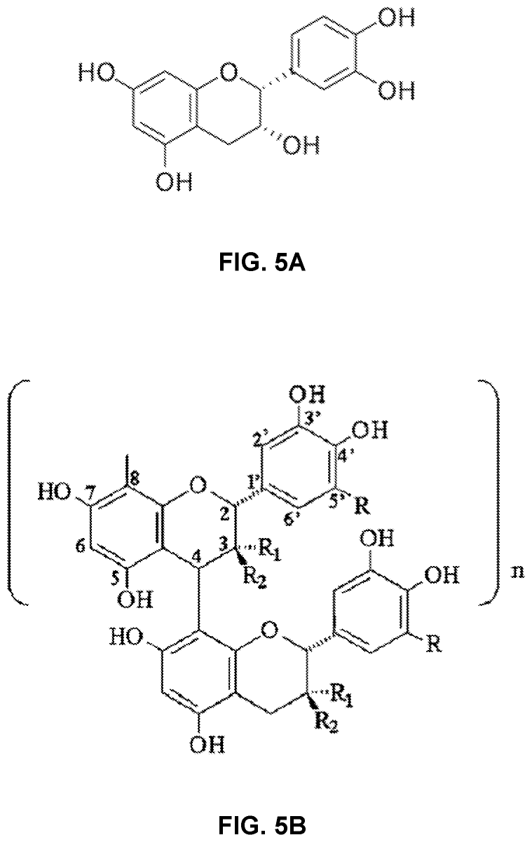

Cranberry commonly contains quercetin and myricetin (FIG. 4) as two flavanols, and quercetin is typically found in relatively high concentrations compared to other fruits. Flavonols are also typically glycosylated. Various metals will chelate quercetin (Symonowicz and Kolanek, 2012), including zinc (Refat et al., 2021).

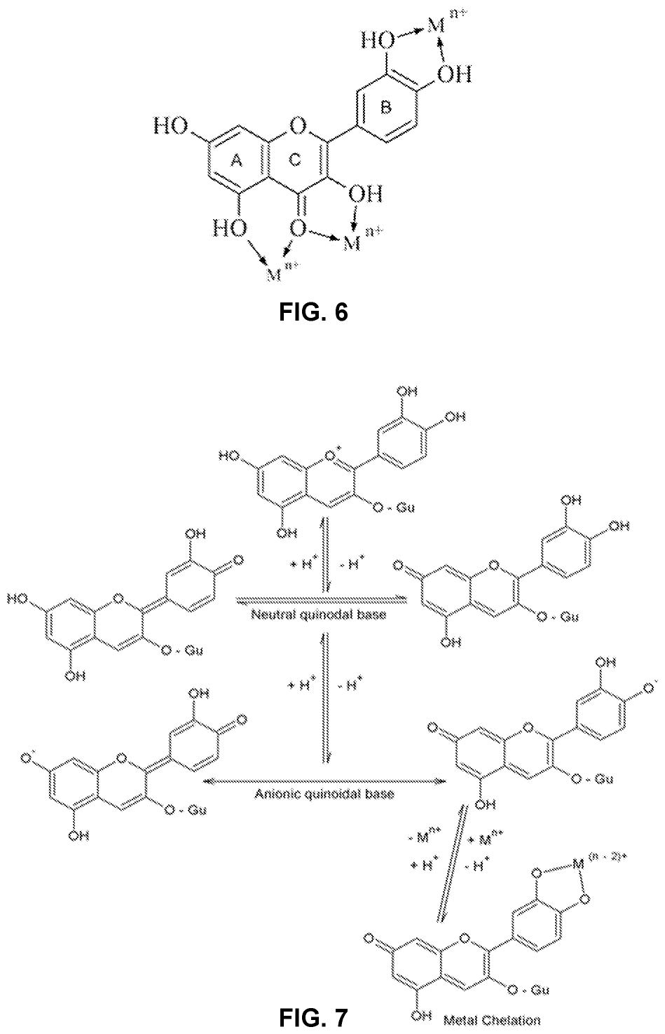

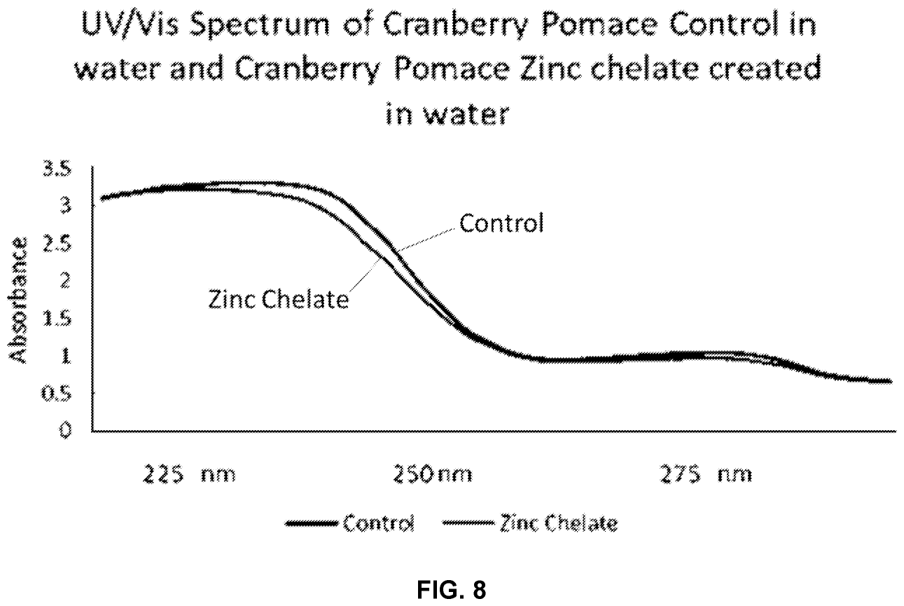

Cranberries also contain relatively high amounts of procyanidins. Procyanidins from cranberry are thought to have anticancer properties and inhibition of bacterial adhesion to the urinary tract and human gastric mucus. Procyanidins are another class of polymeric flavonoids composed of lavan-3-ol units that have antioxidant properties (FIG. 5A). They are also able to chelate metal ions such as zinc, which may further contribute to their health benefits (Luna et al., 2020). Proanthocyanidins (FIG. 5B) in cranberries are health-promoting, but their usages have certain issues (Rauf et al., 2019). Only the monomers and smaller oligomeric procyanidins are known to be absorbed. Proanthocyanidins with a degree of polymerization over 4 are not known to be absorbable due to their large molecular size and gut barrier. Being chelated to zinc may help their bioavailability.

There are practical problems associated with using anthocyanidins, flavanols, and proanthocyanidins for boosting the immune system. Although flavonoid compounds have various pharmacological applications, they generally exhibit low oral bioavailability due to their poor aqueous solubility. They also may not be very stable when exposed to oxygen or elevated temperatures. These characteristics have limited their use in pharmaceutical preparations.

The complexation of metals to whole cranberry pomace (or other semi-processed fruit compositions) has not been explored previously for increasing stability, bioavailability, and boosting the immune system and thus represents a novel solution to these problems. Indeed, unaltered cranberry pomace has not been shown to be very effective for production as a growth promoter in chickens (Leusink et al., 2010). The concurrent complexation of all the components in pomaces simultaneously to metals has also not been carried out previously. Complexing a metal such as zinc to pomace components simultaneously therefore represents an advance in the state of the art for the formation of molecular complexes. Bonding of pomace components through a metal bridge by chelation bonding allows for a synergistic increase in their representative efficacies for antioxidant capability and representative immune stimulating capability. These complexed molecules of the invention solve the problems of stability and bioavailability of pomace components, such as flavonols, anthocyanidins, and proanthocyanidins, to allow for a single pomace to have the health effects of the individual components to synergistically occur efficiently.

When viruses infect cells, one of the immune system's responses is to create interferon that will, in turn, help eliminate viruses. Besides cranberries, certain nutraceuticals can help boost type 1 interferon levels in the cell. Elderberry (Sambucus) extracts containing anthocyanins, ferulic acid, N-acetyl cysteine, Spirulina extracts, selenium, lipoic acid, yeast beta glucans, glucosamine are expected to boost induction of type 1 interferon (McCarty et al., 2020). As an adjunct, these nutraceuticals can complement the activities of the metal-complexed plant compounds of the invention in boosting the immune system or maintaining optimum immunity on a daily basis.

Mycotoxins are toxins that occur naturally, are generated by particular molds (fungi), and appear in food. They can be the source of various negative health effects and can be a significant health danger to livestock, horses, companion animals, and humans. Clays can be added to feedstocks to bind mycotoxins and help prevent or reduce the effects of mycotoxins. Aflatoxins are amongst the most poisonous mycotoxins and are produced by certain molds which grow in soil, decaying vegetation, hay, and grains. The metal-complexed plant compounds of the invention may be combined with aflatoxin binding agents such as clays or styrene-divinylbenzene resins (e.g., Amberlite™ XAD®-2 (Millipore Sigma)) for use in production animals to increase gain. This would occur via synergistic effects of removing toxins while providing for antioxidant and immune stimulation.

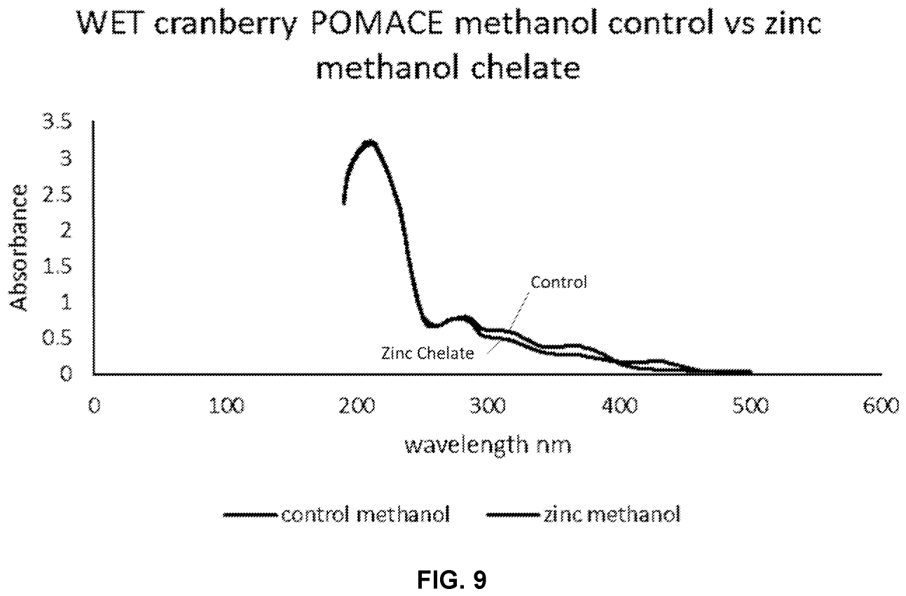

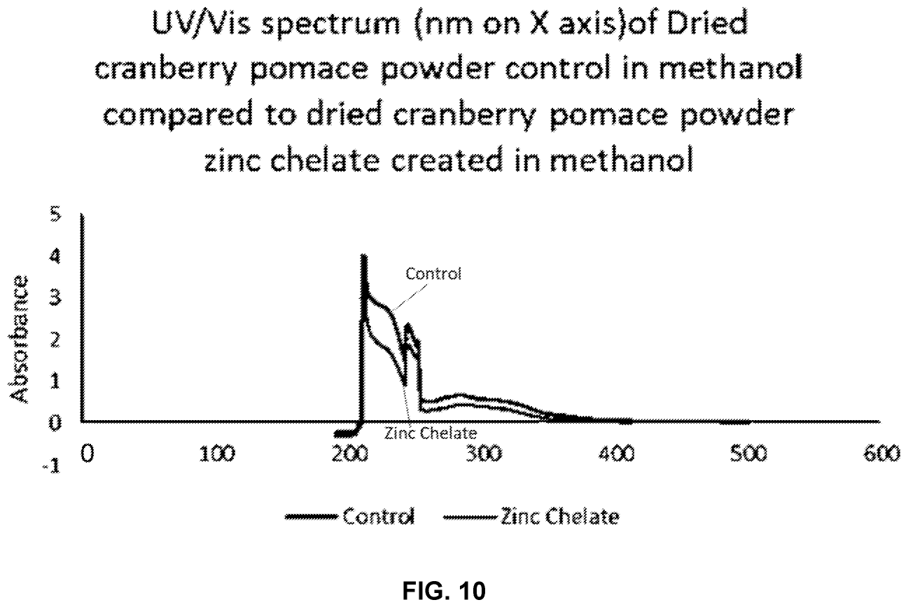

The metal-complexed plant compounds of the invention can include flavonol-metal and anthocyanidin-metal complexes. As used herein, flavonol-metal and anthocyanidin-metal complexes refer to compounds having the structures as seen in FIGS. 6 and 7.

The metal-complexed plant compounds can be generated in any of a number of methods. In one exemplary method, a semi-purified plant composition (e.g., a plant pomace, a plant juice, a liquid plant concentrate, a plant extract, or a combination thereof) can be mixed with a metal. In some versions, the semi-purified plant composition and the metal are directly mixed. In some versions, at least one of the semi-purified plant composition and the metal is mixed with a solvent before being mixed with each other. In some versions, the semi-purified plant composition is first mixed with a solvent before being mixed with the metal. In some versions, the metal is first mixed with a solvent before being mixed with the semi-purified plant composition. In some versions, the semi-purified plant composition and the metal is mixed with a solvent before being mixed with each other. Suitable solvents include water, methanol, ethanol, glycerol, propylene glycol, or others. In some versions, 0.5 parts by mass (e.g., 0.5 g) of the pomace is mixed with 0.5 parts by mass (e.g., 0.5 g) of the metal. This gives 1 part of total pomace ligand with each metal ligand in a 1:1 ratio by mass. Other relative ratios of semi-purified plant composition to metal can be used depending on the requirements of the final product. Exemplary pomace to metal ratios (by mass) include 100:1, 75:1, 50:1, 25:1, 10:1, 7:1, 3:1, 1:1, 1:3, 1:7, 1:10, 1:25, 1:50, 1:75, and 1:100, any ranges between these exemplary ratios, or any and ratios or ranges above or below these exemplary ratios.

The mixed metal and semi-purified plant composition can be stirred at room temperature or higher for 2 hours or longer as required for complexation to occur.

Any solvent can then be removed from the metal-complexed plant compounds by filtration or other means. The residue can be washed in water as required to remove unreacted metal, and further dried to create a powder. Analysis of the complex can be carried out to quantify the metal content of the complex. Ultraviolet (UV) methods can be used to determine that complexation has occurred by comparing to control semi-purified plant composition.

The powder can be mixed with other components, such as anti-inflammatory agents, immune boosting agents, antioxidants, phytobotanicals, and/or carriers.

In some versions, methods of making the metal-complexed plant compounds of the invention can comprise: (1) mixing a semi-purified plant composition (e.g., a plant pomace, a plant juice, a liquid plant concentrate, a plant extract, or a combination thereof) with a first solvent to form a first solution; (2) mixing a metal with a second solvent to form a second solution; (3) mixing the first solution with the second solution to thereby form a mixed solution; and (4) incubating the mixed solution for a time and a temperature suitable for generating the metal-complexed plant compounds.

In some versions, the methods of making the metal-complexed plant compounds of the invention can comprise incubating a semi-purified plant composition (e.g., a plant pomace, a plant juice, a liquid plant concentrate, a plant extract, or a combination thereof) and a metal in a solvent for a time and a temperature suitable for generating the metal-complexed plant compounds. The methods can further comprise, after removing the solvents and unreacted metal, drying the metal-complexed plant compounds into a powder. In some versions, the pomace and the metal are mixed in dry form prior to mixing with the solvent. In some versions, the semi-purified plant composition and the metal are each added separately in dry form to the solvent.

The metal-complexed plant compounds of the invention can be included in a complexed composition suitable for administration to an animal. The complexed composition can be suitable for oral administration. The complexed compositions can be in a solid form, a liquid form, or a semi-solid form (e.g., gel, paste, cream). Exemplary solid forms include powder, tablets, etc.

The metal-complexed plant compounds of the invention can be included in the metal-complexed compositions in an amount from 0.0000000001% w/w to 99.999999999% w/w.

Exemplary amounts include 0.1% w/w, 5% w/w, 10% w/w, 15% w/w, 20% w/w, 25% w/w, 30% w/w, 35% w/w, 40% w/w, 45% w/w, 50% w/w, 55% w/w, 60% w/w, 65% w/w, 70% w/w, 75% w/w, 80% w/w, 85% w/w, 90% w/w, or 95% w/w; any range between these amounts, or any amount or range above or below these amounts.

The complexed compositions can include any of a number of additional components, such as inert carriers and/or active ingredients. Each additional component can be included in the complexed composition in an amount from 0.0000000001% w/w to 99.999999999% w/w. Exemplary amounts include 0.1% w/w, 5% w/w, 10% w/w, 15% w/w, 20% w/w, 25% w/w, 30% w/w, 35% w/w, 40% w/w, 45% w/w, 50% w/w, 55% w/w, 60% w/w, 65% w/w, 70% w/w, 75% w/w, 80% w/w, 85% w/w, 90% w/w, or 95% w/w; any range between these amounts, or any amount or range above or below these amounts. “Active ingredients” can include ingredients other than the metal-complexed plant compounds of the invention having therapeutic or other value when administered to an animal, such as drugs, nutrients, cosmeceuticals, diagnostic agents, nutritional agents, and the like. Exemplary types of active ingredients include anti-inflammatory agents, antioxidants, antimicrobial agents, and immune boosters. Exemplary antioxidants include phytobotanicals, anthocyanins, polyphenols, ferulic acid, N-acetyl cysteine, Spirulina extracts, selenium, lipoic acid, yeast beta glucans, and glucosamine. Exemplary antimicrobial agents include phytobotanicals. Exemplary immune boosting agents include zinc and selenium. Exemplary inert carriers include water or other inert carriers known in the art.

The amounts of each of the components in the final product may be varied depending upon the nature of the additional components, the weight and condition of the animal to be treated, and the unit dosage desired. Those of ordinary skill in the art will be able to adjust dosage amounts as required.

Exemplary forms of the complexed compositions of the invention include soft chews, powders, capsules, tablets, boluses, pastes, liquids, foods, animal feeds, candies, drinks, pastes, liquids, and creams. Exemplary compositional forms suitable for oral administration include tablets, boluses, pastes, capsules, liquids, such as human foods, animal feeds, candy, soft chews, or drinks. Exemplary compositional forms suitable for topical administration include pastes, liquids, gels, or creams.

The components of the invention can be placed into capsules, boluses, and tablets for oral administration or in pastes, liquids, gels, or creams for topical administration in times of oxidative stress from diseases or to maintain optimum immunity health on a daily basis. The components of the invention may also be placed into human foods and animal feeds including cereals, soups, liquid drinks, candy, pharmaceuticals aids including band aids, skin care beauty creams, and pastes.

Some versions of the invention include the metal-complexed plant compounds in combination with any one or more, in any combination, of black elderberry (a phytonutrient supporting immune function and promoting a healthy respiratory system), selenium (a micronutrient that helps fight cellular damage), vitamin D3 (builds immunity in times of stress), vitamin C (ascorbic acid, an antioxidant for maintaining production of white blood cells), phycocyanin (a Spirulina extract that provides support by helping to maintain normal interferon levels during oxidative stress), ferulic acid (an organic compound with antioxidant properties that support the body during stress), and piperine. These components can be included in powder form. The components can be encapsulated in a capsule or provided in any other form provided herein.

The complexed compositions can be used in methods of reducing stress, reducing oxidative stress, maintaining health, improving health, improving immunity, improving vaccination efficacy, increasing feed conversion rate, or decreasing pro-inflammatory cytokine levels in an animal (such as a mammal, human, poultry, etc.). The methods can comprise administering a complexed composition of the invention to the animal in a therapeutically effective amount. The therapeutically effective amount can include an amount effective to reduce stress, reduce oxidative stress, maintain health, improve health, improve immunity, improve vaccination efficacy, increase feed conversion rate, or decrease pro-inflammatory cytokine levels in an animal.

The animal in some versions can include an animal suffering from any condition or disease described herein. Exemplary diseases include necrotic enteritis, cancer, arthritis, inflammatory conditions, cardiovascular conditions, neurological conditions, pulmonary conditions, diabetes, hepatic conditions, gastrointestinal conditions, and an infectious disease, and other conditions that place stresses on the body. Exemplary cardiovascular conditions comprise coronary artery disease, stroke, heart failure, hypertensive heart disease, rheumatic heart disease, and cardiomyopathy, among others. Exemplary neurological conditions comprise diseases of the central and peripheral nervous system, including the brain, spinal cord, cranial nerves, peripheral nerves, nerve roots, autonomic nervous system, neuromuscular junction, and muscles. These conditions include epilepsy, Alzheimer disease and other dementias, cerebrovascular diseases including stroke, migraine and other headache disorders, multiple sclerosis, Parkinson's disease, neuroinfections, brain tumors, traumatic disorders of the nervous system due to head trauma, and neurological disorders as a result of malnutrition. Exemplary pulmonary conditions include asthma, chronic obstructive pulmonary disease (COPD), chronic bronchitis, emphysema, lung cancer, cystic fibrosis/bronchiectasis, pneumonia, pleural effusion, and COVID-19. Exemplary forms of diabetes include type 1 diabetes and type 2 diabetes. Exemplary hepatic conditions comprise hepatitis A infection, hepatitis B infection, and hepatitis C infection, fatty liver disease, cirrhosis, liver cancer, and inherited diseases such as hemochromatosis and Wilson disease. Exemplary gastrointestinal conditions comprise celiac disease, irritable bowel syndrome, and irritable bowel disease (Crohn's disease, ulcerative colitis). Exemplary infectious diseases include viral diseases, prion diseases, and bacterial diseases. The animal can include an animal to be or having been vaccinated within a period of 8 weeks, 7 weeks, 6 weeks, 5 weeks, 4 weeks, 3 weeks, 2 weeks, 1 week, 6 days, 5 days, 4 days, 3 days, 2 days, or 1 day. The therapeutically effective amount can be an amount effective to ameliorate the condition or disease or a symptom of the condition or disease.

The animal can comprise any animal. The animal can comprise a human or a non-human animal. The animal can comprise a mammal. Exemplary mammals include humans, bovines, swine, sheep, goats, dogs, cats, primates, apes, monkeys, and rodents. The animal can comprise a bird, such as poultry. The animal can comprise other types of animals, such as fish or reptiles. In some versions, the animal comprises a production animal. Production animals are animals that are farmed for food or products derived therefrom. Exemplary production animals include bovines (including cattle), ovines (including sheep), caprines (including goats), avines (including birds such as poultry), cervines (including deer), porcines (including pigs), reptilians (including reptiles), and piscines (including fish).

The complexed compositions of the invention can be used as a feed additive to help animals (such as production animals) gain weight faster. In some versions, the compositions can serve as an alternative to antibiotics intended for weight gain and growth promotion in production animals. The animal in such versions can comprise any production animal, such as bovines (including cattle), ovines (including sheep), caprines (including goats), avines (including birds such as poultry), cervines (including deer), porcines (including pigs), reptilians (including reptiles), piscines (including fish), or any other production animal. In some versions, the composition is administered in an amount effective to increase feed conversion rate in the animal relative to a control animal not receiving the composition.

In some versions, the composition is administered in an amount effective to decrease serum levels of a pro-inflammatory cytokine in the animal relative to a control animal not receiving the composition. Exemplary pro-inflammatory cytokines include IL-2, IL-8, IL-6, IL-7, IL-15, IL-18, MCP-1, TNFα, and IL-1-beta.

The elements and method steps described herein can be used in any combination whether explicitly described or not.

All combinations of method steps as used herein can be performed in any order, unless otherwise specified or clearly implied to the contrary by the context in which the referenced combination is made.

As used herein, the singular forms “a,” “an,” and “the” include plural referents unless the content clearly dictates otherwise.

Numerical ranges as used herein are intended to include every number and subset of numbers contained within that range, whether specifically disclosed or not. Further, these numerical ranges should be construed as providing support for a claim directed to any number or subset of numbers in that range. For example, a disclosure of from 1 to 10 should be construed as supporting a range of from 2 to 8, from 3 to 7, from 5 to 6, from 1 to 9, from 3.6 to 4.6, from 3.5 to 9.9, and so forth.

All patents, patent publications, and peer-reviewed publications (i.e., “references”) cited herein are expressly incorporated by reference to the same extent as if each individual reference were specifically and individually indicated as being incorporated by reference. In case of conflict between the present disclosure and the incorporated references, the present disclosure controls.

The compositions and methods of the present invention can comprise, consist of, or consist essentially of the essential elements and limitations described herein, as well as any additional or optional components, or limitations described herein or otherwise useful in the art. The invention provided herein suitably may be practiced in the absence of any element which is not specifically disclosed herein.

It is understood that the invention is not confined to the particular construction and arrangement of parts herein illustrated and described, but embraces such modified forms thereof as come within the scope of the claims.

### EXAMPLES

### Example 1

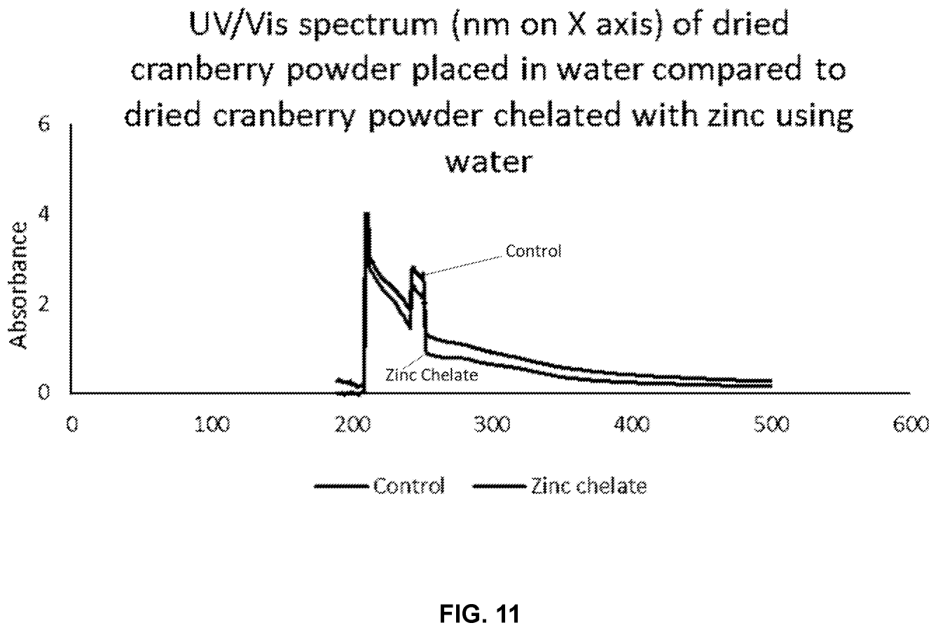

Six grams of ZnCl₂ was dissolved in 40 ml of water in a container. Six grams of cranberry “wet” pomace (70% moisture content) was added to the container and stirred for 1 hour. The entire content of the container was filtered through a glass fiber filter. The supernatant was collected and analyzed on a spectrophotometer for a UV/visible scan from 200 nm to 300 nm. Control material of wet cranberry pomace without ZnCl₂ was stirred in 40 ml water for 1 hour and filtered. The control supernatant was analyzed on a spectrophotometer for a UV/visible scan from 200 nm to 300 nm. The scans were overlayed for comparison (FIG. 8). It is observed that a difference occurs in absorbance at certain wavelength between the control pomace and the zinc-pomace chelate. This indicates zinc is chelating actives such as flavonoids found in the cranberry pomace.

### Example 2

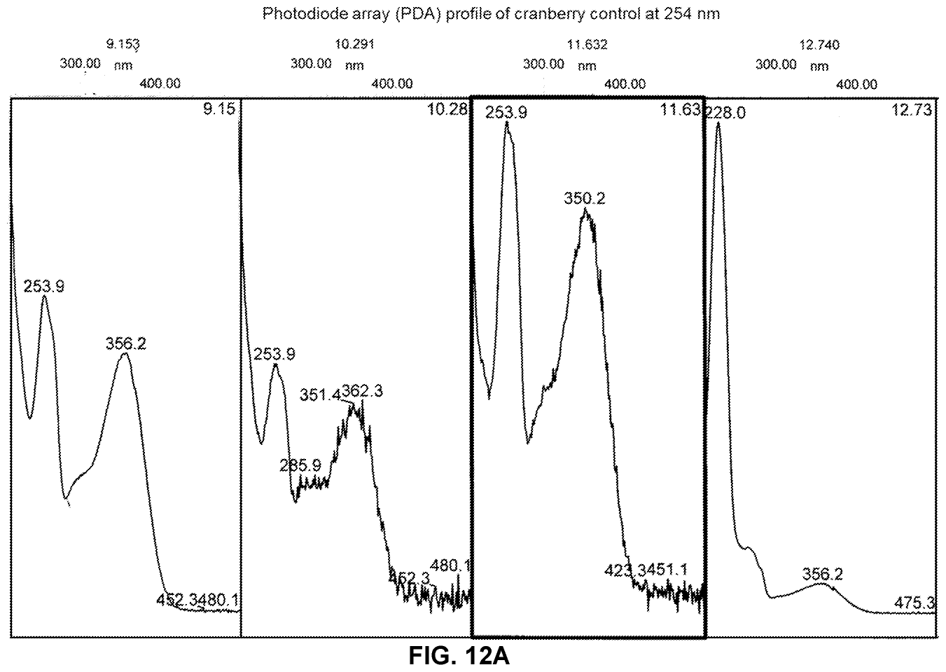

Six grams of ZnCl₂ was dissolved in 40 ml of methanol in a container. Six grams of cranberry “wet” pomace (70% moisture content) was added to the container and stirred for 1 hour. The entire content of the container was filtered through a glass fiber filter. The supernatant was collected and analyzed on a spectrophotometer for a UV/visible scan from 190 nm to 500 nm. Control material of wet cranberry pomace without ZnCl₂ was stirred in 40 ml methanol for 1 hour and filtered. The control supernatant was analyzed on a spectrophotometer for a UV/visible scan from 190 nm to 500 nm. The scans were overlayed for comparison (FIG. 9). It is observed that a difference occurs in absorbance at certain wavelength between the control pomace and the zinc-pomace chelate created in methanol. The profiles of methanol created cranberry pomace-zinc chelates appear different than ones made in water at respective wavelengths. This is consistent with zinc methanol chelating different actives such as proanthocyanidins found in the cranberry pomace than zinc water.

### Example 3

Wet cranberry pomace (10 grams) of 70% moisture content was placed in a mixing bowl and an overhead stirring paddle was run at 60 rpm. Dry ZnCl₂ (1 gram) was dissolved in 10 ml of 1:1 ratio of water to methanol (v:v) and slowly added to commence chelation of the zinc to the pomace. The material was mixed for 1 hour. The chelated material was removed and dried at 50° C. until moisture content was 5%. The chelated dried material was ground into a powder.

### Example 4

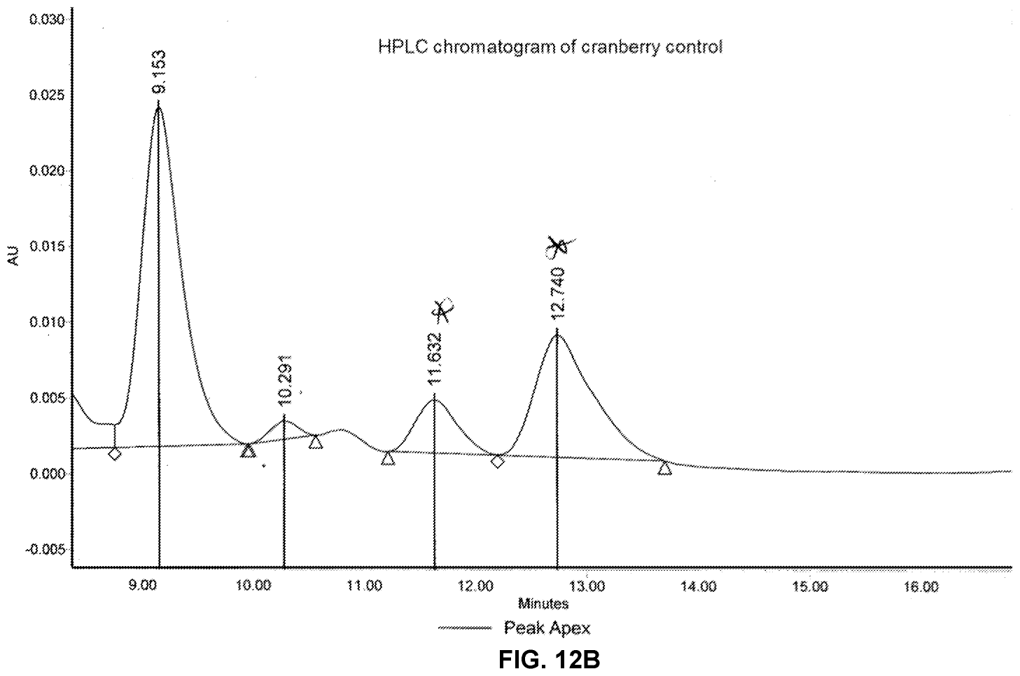

Six grams of dried cranberry pomace powder (5% moisture content) obtained from a different source of cranberries than that used in Examples 1-3 was placed in 40 ml of methanol, 6 grams of ZnCl₂ was added, stirred, and the supernatant was UV analyzed and compared to control powder cranberry pomace that was solvated equally but without zinc (FIG. 10). There are differences in absorbance at different wavelengths between the control and the zinc chelated pomace and the basic profiles are different between sources of cranberry when using methanol. That is consistent with different sources of cranberries having different amounts or types of flavonoids.

### Example 5

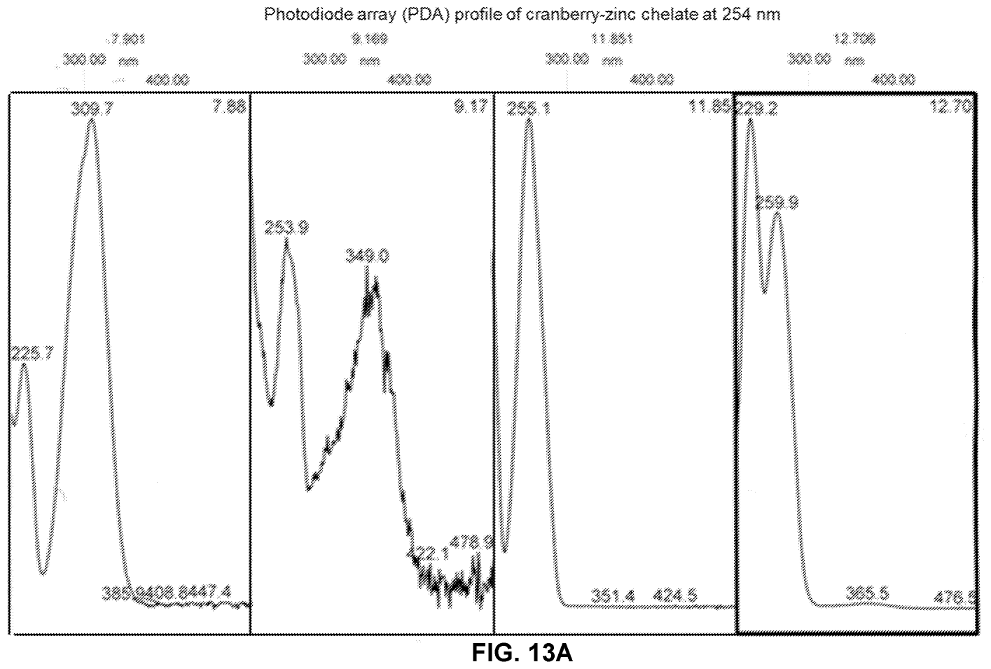

Six grams of dried cranberry pomace powder (5% moisture content) obtained from a different source of cranberries than that used in Examples 1-3 was placed in 40 ml of water, 6 grams of ZnCl₂ was added, stirred, and the supernatant was UV analyzed and compared to control powder cranberry pomace that was solvated equally but without zinc (FIG. 11). There are differences in absorbance at different wavelengths between the control and the zinc chelated pomace and the basic profiles are different between sources of cranberry when using methanol or water. That is consistent with different sources of cranberries having different amounts or types of flavonoids that get chelated differently depending on what the source of cranberry is and whether water or methanol is used to solvate and chelate the zinc to the actives.

### Example 6

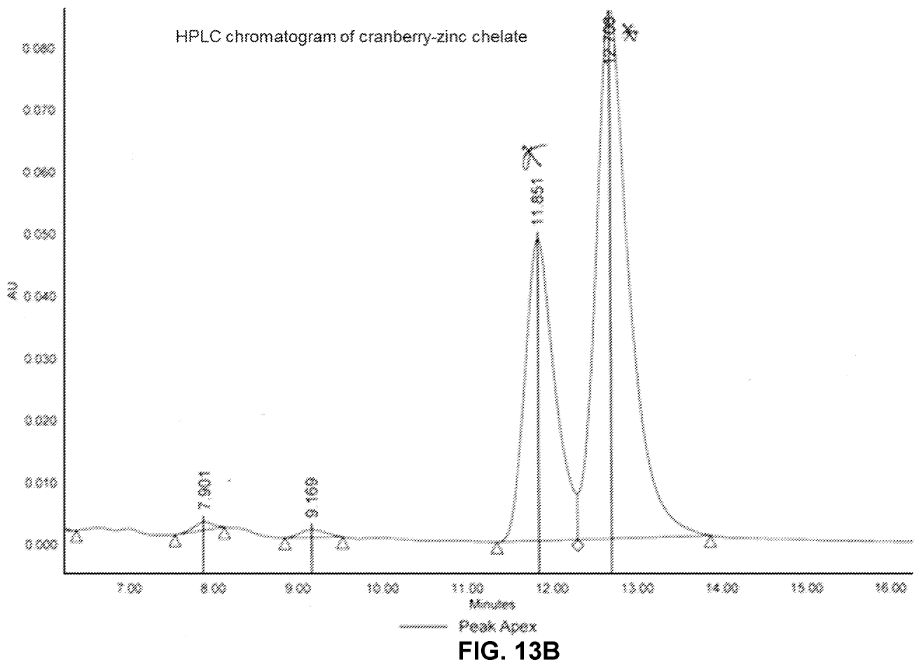

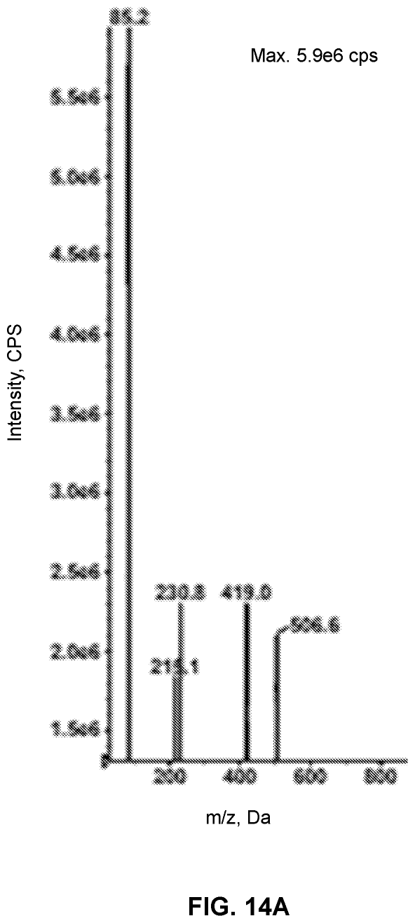

100 ml of a liquid cranberry concentrate was placed in a 200-ml Erlenmeyer flask containing a magnetic stir bar. 10 grams of dry ZnCl₂ was added and mixed for 30 minutes to allow chelation to occur of the zinc to components in the liquid cranberry concentrate. Aliquots of the control cranberry concentrate (FIGS. 12A and 12B) and chelated cranberry (FIGS. 13A and 13B) concentrate were subjected to HPLC analysis using 254 nm for chromatogram profiles. The HPLC results (FIGS. 12B and 13B) show significant differences between the peak heights of the control cranberry and the chelated cranberry at the peaks located at retention times of 9.153, 11.632 and 12.743 minutes. In addition, the photo diode array (PDA) profiles of these peaks (FIGS. 12A and 13A) show changes when zinc is added to the cranberry. While the peak located at 9.153 reduced in relative peak height, the PDA did not change significantly. However, the peaks at 11.632 and 12.743 increase in relative peak height and showed dramatic changes in the PDA profiles. The peaks located at 9.153 and 11.632 are consistent with being identified as flavonols, as based on PDA profile comparisons. These unexpected results are consistent with zinc chelating not just a singular compound in cranberries but to multiple compounds such as the flavonol peaks located at 9.153 or 11.632 minutes, or proanthocyanidins or anthocyanidins.

### Example 7

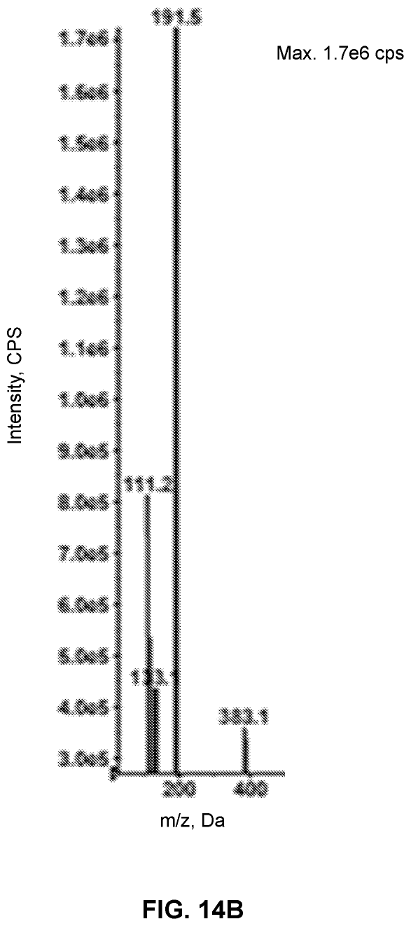

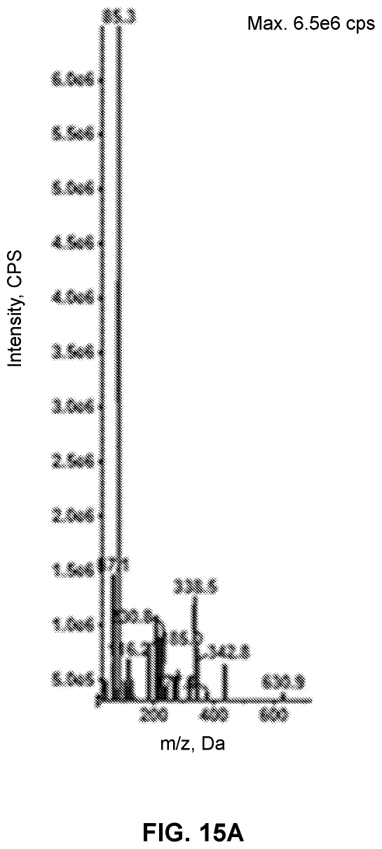

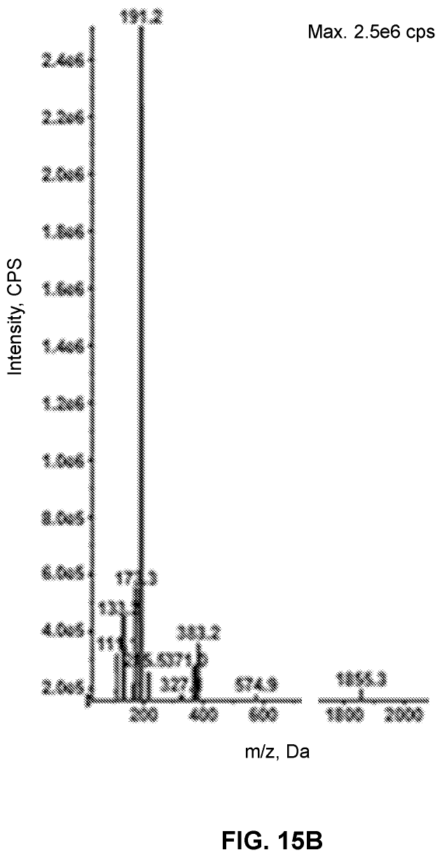

Mass Spectrum (MS) analysis was carried out on the liquid control cranberry concentrate and the zinc-cranberry chelate material (10% w/w) from Example 6. In addition, a second sample of liquid cranberry was chelated using 100 grams of liquid cranberry concentrate with 50 grams of ZnCl₂ (50% w/w). It was observed that large qualitative and quantitative changes in the MS profiles occurred among the samples. The control cranberry concentrate (FIGS. 14A and 14B) contained relatively few peaks, but when zinc was added at 10% w/w (FIGS. 15A and 15B) many more peaks occurred and with larger molecular weights. When 50% w/w ZnCl₂ was added (FIGS. 16A and 16B), even more peaks occurred. For example, no MS peaks were observed in the control material with a m/z greater than 506.6 (FIGS. 14A and 14B). However, when 10% w/w zinc was added to the control cranberry (FIGS. 15A and 15B), m/z peaks are noted at 574, 630.9, 1855.3. These results are consistent with the chelation of zinc to cranberry compounds to increase their mass size. Likewise, when 50% w/w ZnCl₂ was added (FIGS. 16A and 16B), even more overall peaks and different larger m/z compounds, such as 919.6, were noted as compared to 10% zinc-cranberry and the starting control cranberry material. These results are consistent with zinc chelating to small mass sized compounds in the control cranberry matrix to create larger mass size chelation complexes found in the compositions of the invention.

### Example 8

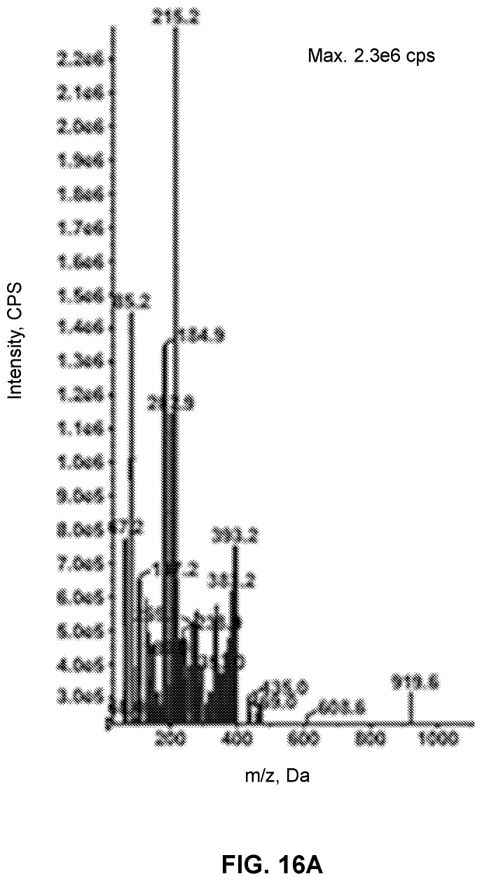

Other metals besides zinc can be used in the compositions of the invention to chelate to plant compounds (e.g., fruits such as cranberry concentrates, cranberry extracts, and cranberry pomace). This is exemplified where 100 g of liquid cranberry concentrate was mixed with 10 g of FeCl₂ or 10 g of FeCl₃ for a ratio of 10% w/w of the metal salts to liquid in a 200-ml Erlenmeyer flask with a polytetrafluoroethylene-coated stir bar. The solution was mixed for 30 minutes to allow chelation to occur and then analyzed for the respective UV profiles of the Fe³⁺-cranberry complexes, Fe²⁺-cranberry complexes and starting control cranberry liquid on a UV-Vis spectrophotometer. We discovered that Fe²⁺ chelation cranberry mixture was very close to the starting control cranberry for the UV profile. Unexpectedly, the Fe³⁺ chelation cranberry mixture showed very large changes in the UV profile compared to the control cranberry (FIG. 17). These results are consistent with the trivalent form of iron in FeCl₃ (ferric chloride) chelating far more effectively than the divalent ferrous chloride to the components in cranberry, such as flavonoids, proanthocyanidins, or other compounds. The use of orally delivered iron-cranberry chelated matrixes may present a novel manner to present iron to piglets without the use of needle injections, along with delivery of other immune boosting positive actives at an early stage of production.

### Example 9

The bioavailability of the compositions of the invention was examined in chickens and compared to the starting control material. 100 grams of liquid cranberry concentrate was chelated with 50 grams of ZnCl₂ as described in Example 7 to generate a 50% w/w mixture. 5 ml of this chelated composition was orally given to a 1-year-old Rhode Island Red chicken via a syringe to fully ensure consumption of the composition. 5 ml of the starting liquid cranberry concentrate (control) was orally given to another 1-year-old Rhode Island Red chicken. At 1 and 2 hours post oral delivery of the chelated composition and the control, blood was removed from the chickens and allowed to clot. Blood was also removed from a 1-year-old Rhode Island Red chicken that did not receive either the chelated composition or the control composition. The serum was analyzed by HPLC. The 1 hour results showed that a peak at 21.259 minutes retention time for the chelated composition had a peak area of 52488, whereas the peak area of the control had a peak area of 6730, while no peak at 21.259 minutes was observed in the serum of the chicken that received neither the chelated composition nor the control. No peaks at 21.249 minutes were observed 2 hours post oral delivery in any of the chickens, indicating complete metabolization of the compounds of the orally chelated composition and control by 2 hours. These results indicate that the compositions of the invention gives over a 7× increase bioavailability of at least one substance found in cranberry concentrates, which is subsequently metabolized and disappears from the serum. These results are consistent with improved efficacy of the compositions of the invention over the starting cranberry concentrates alone.

### Example 10

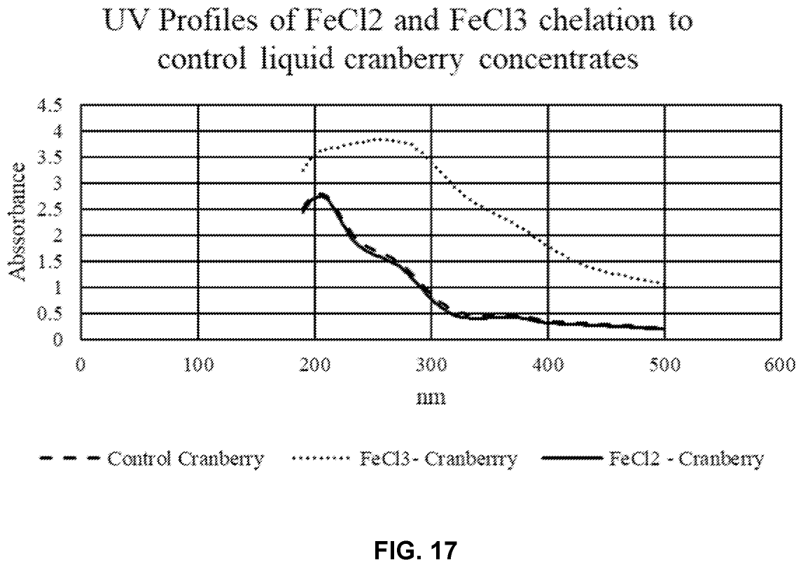

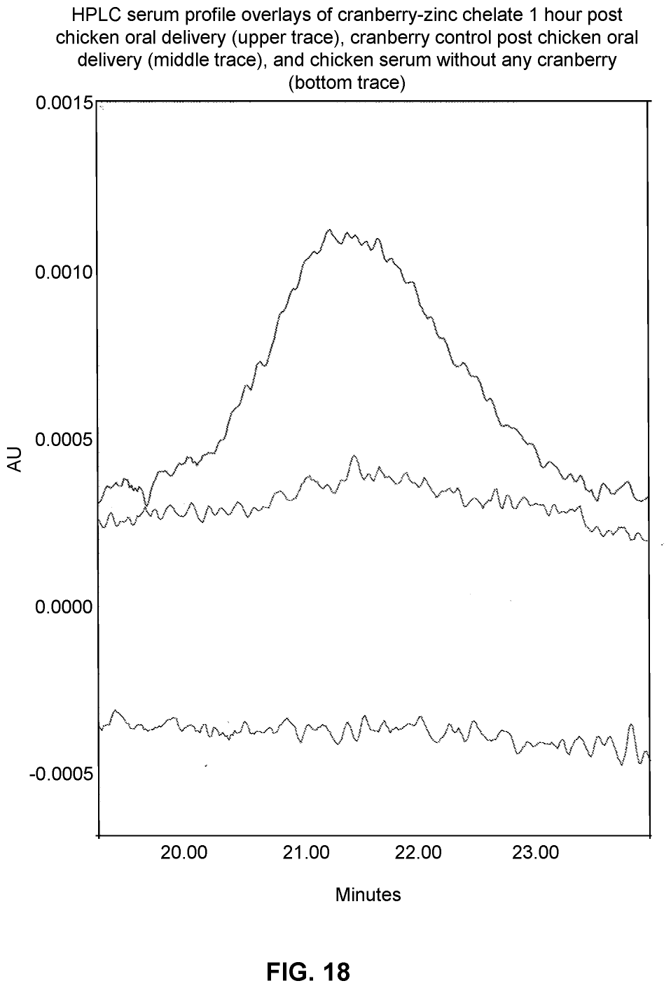

A feed trial for growth promotion was carried out with a composition of the invention made with pomace and zinc as described in Example 3 (pomace-zinc composition) and compared to control chickens that did not receive the pomace-zinc composition. 1 day old Cornish Rock cross chickens (n=10) were fed standard mash feed over the course of the trial length of 5 weeks. Under identical pen housing conditions for lighting, heat, and water, another group of identical 1 day old chickens (N=10) were given the pomace-zinc composition in their daily feed at a 0.5% w/w (pomace-zinc composition/feed) rate. The amount of feed consumed by each group was recorded weekly. At the end of 5 weeks, the individual chickens were weighed and the total pen weights were recorded (FIG. 19) for the control and cranberry-zinc chickens. A feed conversion rate (FCR) was determined dividing the amount of grams of feed consumed at the end of 5 weeks in that pen (FIG. 20) by the average weight of bird in the pen. The data indicate that the pomace-zinc composition gives a higher overall pen weight than the control pens at the end of week 5 (FIG. 19). This is consistent with the pomace-zinc composition helping to improve the overall health of the birds for growth promotion that results in increased weight in this production model. The pomace-zinc composition also shows a desirable lower FCR value than the controls (FIG. 20), meaning the chickens consuming the pomace-zinc composition had to eat less to achieve more weight. The improved FCR with the pomace-zinc composition will be consistent with its use as an antibiotic-free growth promoter.

### Example 11

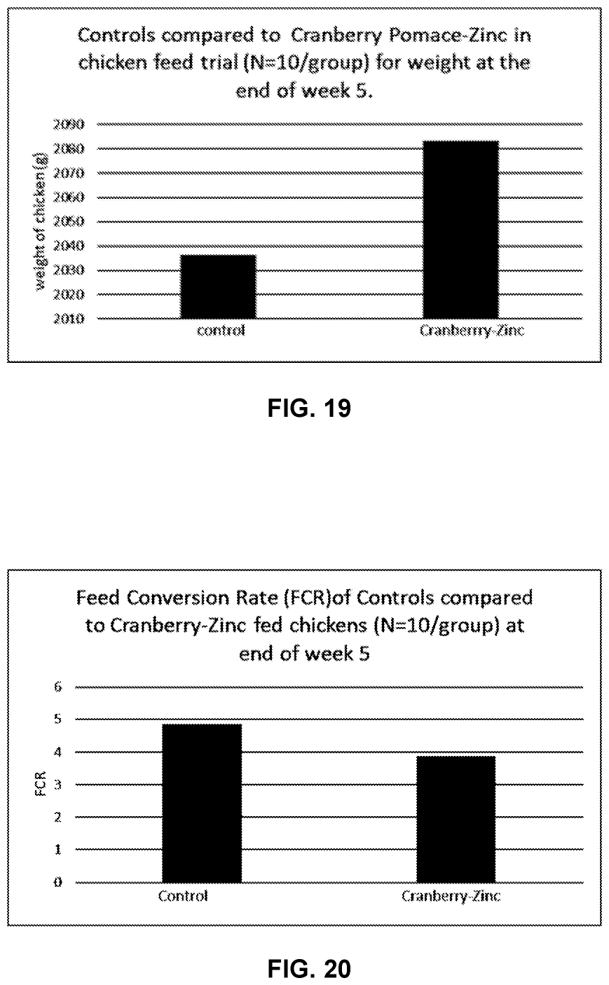

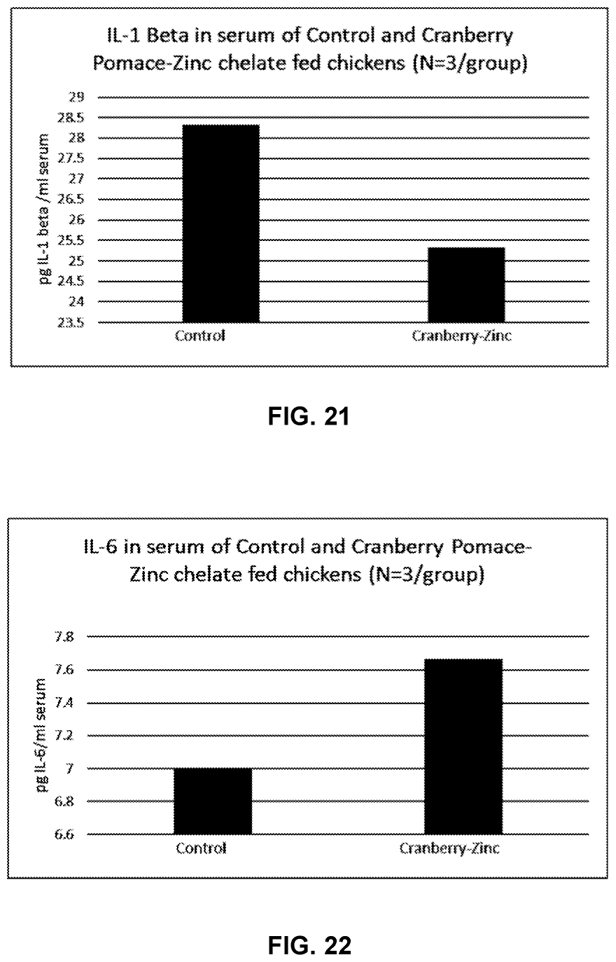

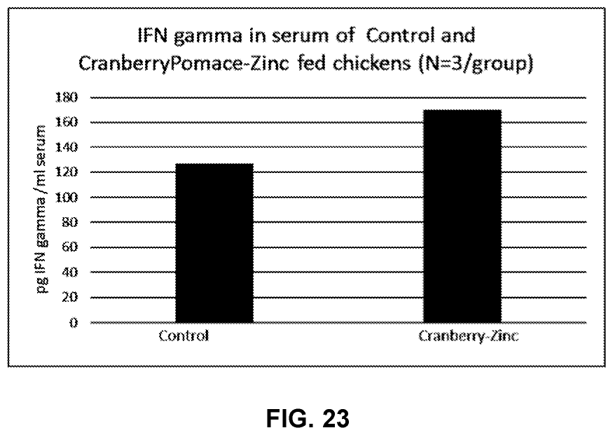

The immune status and levels of pro-inflammatory cytokines and anti-inflammatory cytokines of an animal can be used as a measure of overall health of an animal. Supplements that can modulate cytokine levels in a beneficial manner can be effective in maintaining normal health in times of stress in animals, such as from diseases. To measure the efficacy of the compositions of the invention on cytokine levels, serum from chickens (N=3/group) from Example 10 were analyzed by ELISA using cytokine commercial assays from Cusabio Technology LLC (Houston, TX) for interleukin-1 beta (Il-1 beta), interleukin 6 (IL-6) and interferon-gamma (IFN-gamma). The results showed that the composition of invention reduced the amounts of the pro-inflammatory IL-1 beta in the chickens compared to control chickens (FIG. 21). This would be considered to be beneficial for overall health of the animal. The results showed that amounts of the anti-inflammatory IL-6 increased in chickens receiving the composition of the invention (FIG. 22). The results also showed that amounts of IFN-gamma increased in chickens receiving the composition of the invention (FIG. 23). IFN-gamma is an important cytokine in host defense against pathogens in chickens. These results would be considered beneficial for the health of the animal.

### REFERENCES

Fosmire G J. Zinc toxicity. Am J Clin Nutr. 1990 February; 51 (2):225-7.
GRAS Notice (GRN) No. 873. fda.gov/food/generally-recognized-safe-gras/gras-notice-inventory.
GRAS Notice (GRN)-000719. fda.report/media/110930/Agency-Response-Letter-GRAS-Notice-No.-GRN-000719.pdf
Janeeshma, Edappayil & K Rajan, Vijisha & Puthur, Jos. (2020). Spectral variations associated with anthocyanin accumulation; an apt tool to evaluate zinc stress in Zea mays L. Chemistry and Ecology. 37. 1-18. 10.1080/02757540.2020.1799993.
Luna F. J. C., Mendoza A. M., Balandrán-Quintana W. Antiradical and chelating ability of (+)-catechin, procyanidin B1, and a procyanidin-rich fraction isolated from brown sorghum bran. Nova Scientia Vol. 12 No. 24 (2020) NATURAL SCIENCES AND ENGINEERING doi.org/10.21640/ns.v12i24.2006.
Leusink G., Rempel H., Skura B., Berkyto B. White W., Yang Y., Rhee J. Y., Xuan S. Y. Chiu S., Silversides F., Fitzpatrick S., Diarra M. S. Growth performance, meat quality, and gut microflora of broiler chickens fed with cranberry extract. Poultry Science, Volume 89, Issue 7, 2010, Pages 1514-1523.
McCarty, Mark F, and James J DiNicolantonio. “Nutraceuticals have potential for boosting the type 1 interferon response to RNA viruses including influenza and coronavirus.” Progress in cardiovascular diseases, S0033-0620 (20) 30037-2. 12 Feb. 2020, doi:10.1016/j.pcad.2020.02.007.
Rauf A., Imran M., Abu-Izneid T., lahtisham-Ul-Haq, Patel S., Pan X., Naz S., Silva A. S., Saeed F., Suleria H. A. R. Proanthocyanidins: A comprehensive review, Biomedicine & Pharmacotherapy, Volume 116, 08999, 2019, ISSN 0753-3322, doi.org/10.1016/j.biopha.2019.108999.
Refat M. S., Hamza R. Z., Abdel Majid A. Adam, Hosam A. Saad, Adil A. Gobouri, Fatimah S. Al-Harbi, Fawziah A. Al-Salmi, Tariq Altalhi, Samy M. El-Megharbel. Quercetin/Zinc complex and stem cells: A new drug therapy to ameliorate glycometabolic control and pulmonary dysfunction in diabetes mellitus: Structural characterization and genetic studies. Published: Mar. 4, 2021 Plos One doi.org/10.1371/journal.pone.0246265
Ribeiro T B, Oliveira A, Coelho M, Veiga M, Costa E M, Silva S, Nunes J, Vicente A A, Pintado M. Are olive pomace powders a safe source of bioactives and nutrients? J Sci Food Agric. 2021 Mar. 30; 101 (5):1963-1978. doi: 10.1002/jsfa.10812. Epub 2020 Oct. 6. PMID: 32914435.
Rodríguez-Pérez C, Quirantes-Piné R, Contreras Mdel M, Uberos J, Fernández-Gutiérrez A, Segura-Carretero A. Assessment of the stability of proanthocyanidins and other phenolic compounds in cranberry syrup after gamma-irradiation treatment and during storage. Food Chem. 2015 May 1; 174:392-9. doi: 10.1016/j.foodchem.2014.11.061. Epub 2014 Nov. 18. PMID: 25529697.
Shoba G, Joy D, Joseph T, Majeed M, Rajendran R, Srinivas P S. Influence of piperine on the pharmacokinetics of curcumin in animals and human volunteers. Planta Med. 1998 May; 64 (4):353-6.
Skinner R C, Gigliotti J C, Ku K M, Tou J C. A comprehensive analysis of the composition, health benefits, and safety of apple pomace. Nutr Rev. 2018 Dec. 1; 76 (12):893-909. doi: 10.1093/nutrit/nuy033. PMID: 30085116.
Symonowicz M. Kolanck M. Flavonoids and their properties to form chelate complexes. Biotechnol Food Sci. 2012. 76 (1), 35-41
White B. L. Howard L. R. and Prior R. L. Proximate and Polyphenolic Characterization of Cranberry Pomace. J. Agric. Food Chem. 2010. 58. 4030-4036. DOI:10.1021/jf902829g.

1. A complexed composition comprising a metal-complexed plant compound comprising a plant compound complexed with a metal, wherein the metal-complexed plant compound is generated by a process comprising mixing the metal with a semi-purified plant composition that comprises the plant compound to thereby complex the plant compound with the metal and generate the metal-complexed plant compound.

2. The complexed composition of claim 1, wherein the semi-purified plant composition comprises a plant pomace, a plant juice, a liquid plant concentrate, a plant extract, or a combination thereof.

3. The complexed composition of claim 1, wherein the semi-purified plant composition is derived from a fruit or vegetable.

4. The complexed composition of claim 1, wherein the plant compound comprises a polyphenol.

5. The complexed composition of claim 1, wherein the plant compound comprises a flavonoid.

6. The complexed composition of claim 1, wherein the metal is a divalent metal or a trivalent metal.

7. The complexed composition of claim 1, wherein the metal is zinc, iron (III) or a combination thereof.

8. The complexed composition of claim 1, wherein the metal is mixed with the semi-purified plant composition in an amount of at least 0.1% w/w of the semi-purified plant composition.

9. The complexed composition of claim 1, wherein the process comprises mixing the metal with the semi-purified plant composition in a solvent.

10. The complexed composition of claim 9, wherein the solvent comprises water, an alcohol, or a combination thereof.

11. The complexed composition of claim 1, wherein the complexed composition further comprises an active ingredient extraneous to the semi-purified plant composition.

12. The complexed composition of claim 11, wherein the active ingredient comprises at least one of an antioxidant, an antimicrobial agent, and an immune-boosting agent.

13. The complexed composition of claim 1, wherein the active ingredient comprises one or more of a phytobotanical, an anthocyanin, a polyphenol, ferulic acid, N-acetyl cysteine, a Spirulina extract, a clay, and styrene-divinylbenzene resin.

14. The complexed composition of claim 1, wherein the complexed composition is in the form of a powder.

15. The complexed composition of claim 1, wherein:
the semi-purified plant composition comprises a plant pomace, a plant juice, a liquid plant concentrate, a plant extract, or a combination thereof;
the semi-purified plant composition is derived from a fruit or vegetable;
the plant compound comprises a flavonoid;
the metal is zinc, iron (III) or a combination thereof;
the metal is mixed with the semi-purified plant composition in an amount of at least 1% w/w of the semi-purified plant composition;
the process comprises mixing the metal with the semi-purified plant composition in a solvent;
the solvent comprises water, an alcohol, or a combination thereof; and
the complexed composition is in the form of a powder.

16. A method comprising administering the complexed composition of claim 1 to an animal.

17. The method of claim 16, wherein the animal is a human, a production animal, or a companion animal.

18. The method of claim 16, wherein the complexed composition is administered to the animal in an amount effective to increase weight of the animal relative to a control animal not administered the complexed composition.

19. The method of claim 16, wherein the complexed composition is administered to the animal in an amount effective to decrease the feed conversion rate of the animal relative to a control animal not administered the complexed composition.

20. The method of claim 16, wherein the complexed composition is administered in an amount effective to decrease serum levels of at least one pro-inflammatory cytokine or increase serum levels of at least one anti-inflammatory cytokine in the animal relative to a control animal not administered the complexed composition.

21. The method of claim 16, wherein the complexed composition is administered in an amount effective to decrease serum levels of at least one of Il-1 beta, IL-2, IL-8, IL-7, IL-15, IL-18, MCP-1, TNFα, in the animal relative to a control animal not administered the complexed composition.

22. The method of claim 16, wherein the complexed composition is administered in an amount effective to decrease at least one of interleukin (IL)-1 receptor antagonist, IL-4, IL-6, IL-10, IL-11, and IL-13.

23. The method of claim 16, wherein the animal is an animal suffering from a condition comprising one or more of iron anemia, cancer, arthritis, an inflammatory condition, a cardiovascular condition, a neurological condition, a pulmonary condition, diabetes, a hepatic condition, a gastrointestinal condition, a viral disease, a prion disease, mycotoxin infection, and an infectious disease.

24. The method of claim 16, wherein the complexed composition is administered orally to the animal.
